SEQUENCE LISTING (2 sequences)

SEQ ID NO: 1 - protein, 410 aa, Comamonas acidovorans
MPVNLSAPVHADLLAINGVRIGVTEAGVRKANRKDLSVFLLDEGASVAGVFTQNRFCAAPVQVSREHLAQTSGAIRAMVINTGNANAGTGADGLARARRTCADLAALLQLKPRQILPFSTGVIMEELPVDRIAAGLPAAIAAAKADNWGTAAEGIMTTDTLPKAFSRAVDIGGKRVSITGISKGAGMIRPNMATMLGFLATDAAIAPELLQPLVKELADGSFNRVTIDGDTSTNDSFVLVATHKAGNAQITSLTSAEGLALKAALLDVSRLLAQAIVRDGEGATKFITVKVEGGKTEAECRLVAYSIAHSPLVKTAFFASDPNLGRILAAVGYASIDDLDQTKIDLYLNDVHVAVNGGRNPAYQEEDGQRVMKQQEIIVRVLLGRGQAEQTVWTCDLSHDYVSINADYRS

SEQ ID NO: 2 - DNA, 2172 bp, Comamonas acidovorans
aagcttaccaactggcgtgaccacctgtccgcgctggactatctgcgccagggcatccacctgcgtggctatgcccagaagcagcccaagcaggaatacaagcgcgaggctttcgagctgttccgccagctcatcgaccaggtcaagaccgaagtcacgcgcgtgctgatgaccgtgcaggtgcagtcgcgcgaacaggtggagcaggccaccgaggccatggagcagcgcagcgcccacagcctggagcacatgacctatggcgctccctcggatggcgacatcggtggctccgtcgaagacgagccgctggagttgcctgaaggcgctcgcgtgggccgcaatgatccctgcccctgcggcagcggcaagaagtacaagcagtgccacggcaagctgtcgtgatgcggccggcgtcgtaagacgccgcaagacacggaaaacggcacggatgcactccgtgccgttttcttttttaaggtcgatgaaatacgtgccgccccggtttgttccgataatggagcgcgtttttctttcactgtaggaagcccctcaatgcctgtgaatctctccgctcccgtgcacgccgacctgttggcgatcaacggcgttcgcatcggcgtcaccgaagccggtgtgcgcaaggccaaccgcaaggacctgtccgtgttcctgctcgacgagggcgcttcggtggcgggtgtgttcacgcagaaccgtttctgcgcagcgcccgtgcaggtcagccgcgagcacttggcccagacgtccggcgccatccgtgccatggtcatcaacacaggcaatgccaatgccggcaccggcgctgacggactggcgcgcgcgcgccgcacctgtgcggatctggctgcgctgctgcagctcaagcccgagcagatcctgccgttctccactggcgtgtacatggaagagctgcccgtggatcgcatcgccgccggcctgccagctgccatcgcagcagccaaggccgacaactggggcacggccgccgaaggcatcatgaccaccgacaccctgcccaaggccttcagccgcgccgtggatattggcggcaagcgtgtgagcatcactggcatcagcaagggtgcaggcatgattcgccccaacatggcgaccatgctgggctttctggccaccgacgccgccatcgcacccgaactgctgcagcctctggtcaaggaactggccgacggatcgttcaaccgcgtgaccatcgacggcgacacgtcgaccaacgactccttcgtgctggtggccacgcacaaggccggcaatgcgcagatcacttcgctgaccagtgccgaaggcctggccctgaaggctgcattgctggatgtgtcgcgcctgctggcccaggccatcgtgcgcgacggcgaaggcgccaccaagttcatcaccgtcaaggtggagggcggcaagaccgaggctgagtgccgcctcgtggcctattccatcgcgcattcacccctggtcaagacggcgttctttgccagcgatccgaacctgggtcgcatccttgcggcggtgggctatgctagcatcgacgatctggaccagaccaagatcgacctgtacctgaacgatgtgcatgtcgcagtcaatggcggccgcaaccctgcctatcaggaggaagacggccagcgcgtgatgaagcagcaggagatcatcgtgcgcgtgctactgggccgagggcaggccgagcagaccgtgtggacctgcgatctgagccacgactacgtgagcatcaacgccgactaccgctcctgactctccacccaccgtgccgccggcagccgcaggccttgtgcctggctgctgccagctttcaatggcggccggcgcgcaaaagctctggcgttggcatgagatccatctcgattcccgggcatatccagcgtcggtggccgtgagatccgtattcgagagacatccatgacattgcccacagaagccgtcgccgcccccttgctgcaactcgtgcagcgcgccgagcagctgatgcagcgcatcgaggccatcctgccccagccgctgcaatcccccgccgactggagtgcctccatcgcctggcgctatcgcaagcgcgccaacggctgcggtttgctggagcccgtgcgccatgtcggcgccatgcagctgtccgacctgcag

(19) United States
(12) Patent Application Publication
(10) Pub. No.: US 2005/0081267 A1
(43) Pub. Date: Apr. 14, 2005

(54) Novel genes for conditional cell ablation

(75) Inventors: Alfred Puhler, Bielefeld (DE); Inge Broer, Roggentin (DE); Guido Kriete, Northeim (DE)

Correspondence Address: BIRCH STEWART KOLASCH & BIRCH, PO BOX 747, FALLS CHURCH, VA, 22040-0747, US

(73) Assignee: Bayer Bioscience N.V., Gent (BE)

(21) Appl. No.: 10/471,370

(22) PCT Filed: Mar. 12, 2002
(86) PCT No.: PCT/EP02/02904

(30) Foreign Application Priority Data
Mar. 12, 2001 (EP) 01200913.0

(51) Int. Cl. C12Q 1/68; C07H 21/04; C12N 9/18; A01H 1/00; C12N 15/82; C12N 5/04

(52) U.S. Cl. 800/288; 435/6; 435/69.1; 435/197; 435/419; 435/468; 536/23.2

(57) ABSTRACT

A Novel deacetylase genes for use in conditional cell ablation are described. These genes are particularly useful for the production of transgenic plants with plant parts which can be destroyed upon treatment with N-acetyl-PPT.

### FIELD OF THE INVENTION

The invention relates to novel DNA molecules encoding a protein having the biological activity of a deacetylase. Preferably these DNA molecules encode a protein having a sequence similarity with a deacetylase as described herein isolated from Comamonas acidovorans. The invention further relates to transgenic plants with plant parts which can be destroyed upon induction, comprising the DNA molecules of the invention under control of tissue-specific promoter.

All documents cited herein are incorporated herein by reference.

### BACKGROUND OF THE INVENTION

Deacetylases are capable of deacetylating acetylated toxins, such as N-acetyl-phosphinothricin (N-Ac-PTC or N-Ac-PPT), intracellularly, whereupon the cytotoxic activity of the toxins is restored (formation of PTC or PTC). U.S. Pat. No. 5,650,310, U.S. Pat. No. 5,668,297, U.S. Pat. No. 5,767,370, and U.S. Pat. No. 5,767,371, describe deacetylase genes, for the production of phosphinothricin or phosphinothricyl-alanyl-alanine, as well as processes for their isolation and use. Furthermore deacetylase genes are disclosed which are isolated from Streptomyces viridochromogenes (dea) and from E. coli (ArgE). The use of such genes for the production of plants with selectively destroyable plant parts is described. More specifically, a method is disclosed for the production of conditionally male-sterile plants. This is achieved by introducing a deacetylase gene, under the control of a tapetum-specific promoter into the genome of a plant. Upon application of N-Ac-PPT to the plant, in the tapetum cells, where the deacetylase is expressed, N-Ac-PPT is converted into PPT, which is toxic to the cells. The cytotoxic activity in the tapetum cells inhibits the development of microspores and renders the plant male-sterile.

WO 98/27201 describes novel genes encoding amino acid deacetylases isolated from Stenotrophomonas sp. and from Comamonas acidovorans and their use in the production of tranagenic plants.

WO 98/13504 describes the use of deacetylase genes for the production of female-sterile plants. WO 98/39462 describes a method of hybrid seed production using conditional female sterility, whereby a plant is made conditionally female-sterile by transformation with the ArgE gene linked to a female-preferential promoter.

EP 98116492 describes the use of the deac system for the modification of plant development. Deacetylase genes placed under control of a meristem specific promoter can be used for inducible modification of developmental characteristics of the plant such as flowering and bolting to improve yield, or influence general plant architecture.

EP 98116493 describes the use of the deac system to obtain pathogen control in plants. Deacetylase genes placed under control of a pathogen-inducible promoter can be used to obtain plants which, upon treatment with N-acetyl-PPT become tolerant to pathogens, through selective destruction of pathogen-infected tissue.

However, the foregoing documents fail to teach or suggest the present invention.

### SUMMARY OF THE INVENTION

The present invention relates to DNA molecules encoding a protein having the biological activity of a deacetylase. More specifically the protein having the biological activity of a deacetylase is a protein having an amino acid sequence with at least 70% to 79%, more preferably 80% to 85%, especially preferably 86% to 90%, most preferably 91% to 95%, sequence identity, more specifically which is 96% to 100% identical to the sequence of SEQ ID No. 1 or a biologically active fragment or variant thereof. Especially preferably the DNA molecule of the invention encodes a protein having the amino acid sequence of SEQ ID No. 1 or a biologically active fragment thereof

Alternately or additionally, the DNA molecule of the present invention has a nucleotide sequence which has a sequence identity of at least 70% to 79%, more preferably 80% to 85%, especially preferably 86% to 90%, most preferably 91% to 95%, sequence identity, more specifically which is 96% to 100% identical to the sequence of SEQ ID No. 2. Especially preferably the DNA molecule of the invention comprises the nucleotide sequence of SEQ ID No. 2.

Additionally or alternatively, the invention relates to a DNA molecule encoding a deacetylase which can be isolated from Comamonas acidovorans, deposited as DSM 11070, which comprises the sequence of SEQ ID No. 2.

The invention further relates to a protein or polypeptide having the biological activity of a deacetylase, which is encoded by a DNA molecule, such as those described above.

The invention further relates to a chimeric gene comprising the DNA molecule of the present invention placed in the same transcriptional unit and under the control of a plant expressible promoter, preferably a tissue-specific promoter.

The invention further relates to a plant having inducibly destroyable plant parts, which plant comprises, stably integrated in its genome, a chimeric gene comprising the DNA molecule of the invention, placed under control of a plant-expressible promoter, preferably a tissue-specific promoter, the plant of the invention being characterized by the fact that, upon treatment with N-acetyl-PPT, the plant tissues in which the promoter directs expression, are destroyed.

The invention further relates to an inducibly male or female-sterile plant, which plant comprises, stably integrated in its genome, the DNA molecule of the invention, placed under control of a male- or female reproductive organ selective promoter, the inducibly male- or female-sterile plant of the invention being characterized by the fact that, upon treatment with N-acetyl-PPT, specific plant tissues of the male- or female reproductive organ are destroyed.

The invention further relates to a process for producing a plant with inducibly destroyable plant tissues or parts, which process comprises:
introducing into a plant cell or tissue a foreign DNA comprising a chimeric gene comprising
   a) the DNA molecule of the invention, encoding a protein or polypeptide having the biological activity of a deacetylase, under the control of
   b) a plant-expressible promoter, which is preferably tissue-specific;
regenerating the plant cell or tissue into a plant
optionally, inducing the destruction of the plant tissue or parts by treatment of the plant with N-acetyl-PPT.

### DETAILED DESCRIPTION

The term “gene” as used herein refers to any DNA sequence comprising several operably linked DNA fragments such as a promoter region, a 5′ untranslated region (the 5′UTR), a coding region (which may or may not code for a protein), and an untranslated 3′ region (3′UTR) comprising a polyadenylation site. Typically in plant cells, the 5′UTR, the coding region and the 3′UTR are transcribed into an RNA of which, in the case of a protein encoding gene, the coding region is translated into a protein. A gene may include additional DNA fragments such as, for example, introns.

The term “chimeric” when referring to a gene or DNA sequence is used to refer to the fact that the gene or DNA sequence comprises at least two functionally relevant DNA fragments (such as promoter, 5′UTR, coding region, 3′UTR intron) that are not naturally associated with each other and/or originate, for example, from different sources. “Foreign” referring to a gene or DNA sequence with respect to a plant species is used to indicate that the gene or DNA sequence is not naturally found in that plant species, or is not naturally found in that genetic locus in that plant species. The term “foreign DNA” will be used herein to refer to a DNA sequence as it has incorporated into the genome of a plant as a result of transformation.

A genome of a plant, plant tissue or plant cell, as used herein, refers to any genetic material in the plant, plant tissue or plant cell, and includes both the nuclear and the plastid (chloroplast and mitochondrial) genome.

A “fragment” of a DNA molecule or protein sequence as used herein refers to a truncated sequence of the original sequence (nucleic acid or amino acid) referred to, which can vary in length but of which the minimum size is sufficient to ensure the (encoded) protein to be biologically active, the maximum size not being critical. A “variant” of a sequence is used herein to indicate a DNA molecule or protein of which the sequence (nucleic or amino acid) is essentially identical to the sequence to which the term refers.

Sequences which are “essentially identical” means that when two sequences are aligned, the percent sequence identity, i.e. the number of positions with identical nucleotides or amino acids divided by the number of nucleotides or amino acids in the shorter of the sequences, is higher than 70%-80%, preferably 81-85%, more preferably 86-90%, especially preferably 91-95%, most preferably 96-100%, more specifically is 100%. The alignment of two nucleotide sequences is performed by the algorithm as described by Wilbur and Lipmann (1983, Proc. Natl. Acad. Sci. U.S.A. 80:726) using a window size of 20 nucleotides, a word length of 4 nucleotides, and a gap penalty of 4. Preferably the DNA molecule of the invention is a sequence which is essentially identical to a coding sequence of a deacetylase from Comamonas acidovorans, which has been adapted for preferred codon usage in plants.

The present invention relates to a DNA molecule or coding sequence encoding a protein having the activity of a deacetylase. A “deacetylase” or a protein with deacetylase activity as used herein refers to a molecule which is capable of deacetylating N-acetyl PPT (which in itself—i.e. in the absence of a deacetylase- is not toxic to plants or plant cells or tissues), to produce PPT which is cytotoxic to plants. Deacetylase activity can be measured by radioactive or non-radioactive assays as described in WO 98/27201 and U.S. Pat. No. 5,668,297. Presence of deacetylase activity in plant tissues can be determined by treatment of the tissue with the substrate N-acetyl-PPT, and evaluating production of PPT, which should result in cell death. A protein or polypeptide which is “biologically active” as used herein refers to the fact that it has deacetylase activity.

In a preferred embodiment of the invention, the DNA molecule encoding a protein having the biological activity of a deacetylase, is a DNA which can be isolated from the deposit with International deposit No. DSM 11070 (as described in WO 98/27201 and U.S. Pat. No. 6,177,616) deposited at the. Deutsche Sammlung für Microorganismen und Zelikulturen GmbH, Masenroder Weg 1b, D-38124. Braunsenweig. More specifically such a DNA molecule is a DNA-molecule encoding a deacetylase having a sequence which is essentially identical to the sequence of SEQ ID No. 1, or a biologically active fragment thereof. Additionally or alternatively such a DNA molecule comprises a nucleotide sequence which is essentially identical to the sequence of SEQ ID No. 2, or corresponds to a DNA sequence which hybridizes with the sequence of SEQ ID No. 2 under standard stringent conditions.

A preferred embodiment of the DNA molecule of the present invention is a DNA molecule encoding a deacetylase comprising a sequence which is essentially identical to SEQ ID No. 1, isolated from Comamonas acidovorans. Based on the homology of the DNA sequence isolated from Comamonas acidovorans to N-acetyl-Ornithine transferase genes, the deacetylases of present invention will be referred to as “ArgJ” deacetylases and the genes comprising a DNA molecule encoding such an ArgJ deacetylase (or a biologically active fragment thereof) will be referred to as an “ArgJ gene”.

The isolation of sequences encompassed by the present invention which are essentially identical to the sequence of SEQ ID No.1 isolated from Comamomas acidovorans, is possible using standard strategies and techniques that are well known in the art. For example, using all or a portion of the amino acid sequence of the Comamonas ArgJ protein, one may readily design specific oligonucleotide probes, including degenerate oligonucleotide probes (i.e., a mixture of all possible coding sequences for a given amino acid sequence). The oligonucleotides may be based upon the sequence of either DNA strand and any appropriate portion of the ArgJ coding sequence. General methods for designing and preparing such probes are provided, for example, in Ausubel et al. Current Protocols in Molecular Biology, Wiley Interscience, New York and Berger and Kimmel, Guide to Molecular Cloning techniques, 1987, Academic Press, New York. These oligonucleotides are useful for ArgJ gene isolation, either through their use as probes capable of hybridizing to ArgJ complementary sequences or as primers for various amplification techniques, for example PCR cloning strategies.

For detection or isolation of closely related ArgJ sequences having greater than 80% sequence identity, high stringency conditions are preferably used; such Stringent conditions as used herein refers to the hybridizing conditions described by Sambrook et al. (1989) (Molecular Cloning: A. Laboratory Manual, Second Edition, Cold Spring Harbour Laboratory Press, NY).

As used herein N-acetyl-PPT refers to N-acetyl-phosphinothricin or a composite structure thereof, which in itself is not toxic to plant cells but is converted to a toxin (phosphinothricin, PPT) by a deacetylase. Thus, the term N-acetyl-PPT refers to any formulation of N-acetyl-phosphinothricin, or derivatives or related compounds, such as acetylated forms of desmethylphosphinothricine (2-amino-4-hydroxyphosphinobutyric acid), bialaphos (phosphinothricyl-alanyl-alanine), desmethylbialaphos and the like, their salts, racemic mixtures and active enantiomers, which can be used in the context of the present invention.

According to the present invention, plant-expressible promoter is a promoter which directs expression in plants. Preferably, the plant-expressible promoter of the invention is a tissue-specific promoter, which is a promoter directing expression selectively in a specific cell or tissue of the plant. A broad range of tissue-specific promoters are described in the literature, such as, but not limited to, meristem-specific promoters (such as the promoter of the AGL8 MADS box gene; Yanofsky, 1995, The plant cell 7:1763-1771), female-organ specific promoters (such as the PSTMG-type promoters described in U.S. Pat. No. 4,633,441), leaf-tissue specific promoters (such as the leaf-specific Ca/b promoter from Arabidopsis described by Cannon et al., 1990, Plant Mol Biol 15(1)39-47 or the tomato RbcS-3C promoter described by Kyozuka et al., 1993, Plant Physiol 102(3):991-1000).

In a preferred embodiment of the invention, the tissue-specific promoter is male or female organ selective promoter, directing expression selectively in male or female organs of the plant. A preferred male organ selective promoter is a stamen-selective promoter which directs expression in selective stamen cells (including anther cells and pollen), preferably directing expression essentially only in stamen cells. More specifically, the stamen-selective promoter is a tapetum-specific promoter directing expression specifically in tapetum cells. A number of stamen-selective promoters are known in the art, such as, but not limited to, the TA29 promoter from tobacco (U.S. Pat. No. 5,652,354), the CA55 promoter isolated form corn (WO 92/00275, U.S. Pat. No. 5,589,610), the T72 and E1 promoters isolated from rice (U.S. Pat. No. 5,639,948), SGBG (U.S. Pat. No. 5,837,850), and 5126 (U.S. Pat. No. 5,689,049).

Tissue-specific promoters as used herein includes promoters which are also subject to other kinds of regulation (such as, but not limited to, time-dependent, stress-dependent, environment-dependent, chemically inducible, etc.).

Alternatively, the plant-expressible promoters of the present invention are not tissue-specific but are subject to other kinds of regulation (such as, but not limited to, time-dependent, stress-dependent, environment-dependent, chemically inducible, etc.).

A plant with conditionally or inducibly destroyable parts refers to a plant in which, under specific conditions, specific plant tissues or parts can be destroyed. “Plant part” is meant to refer to any plant organ (such as, but not limited to male or female reproductive organs, leaves, vegetative meristem, roots) or tissue (such as, but not limited to reproductive tissues, embryogenic tissues), including the complete plant. More specifically, these conditions are understood to refer to the treatment of the plant with an acetylated toxin, which normally is not toxic to the plant or plant cells, but in the presence of a deacetylase in the plant cell is converted into a substance that is cytotoxic.

A conditionally male-sterile plant refers to a plant which, under normal growing conditions, is male fertile, i.e. produces fertile pollen, and which can be made male-sterile under specific conditions. Treatment of the plant can refer to administering the substance in any way, such as, but not limited to manual treatment, or spraying of the plant, plant part (or of the field or greenhouse wherein the plant is grown), or through other forms of irrigation.

Treatment of a plant with N-acetyl PPT will result in uptake of N-acetyl PPT by the leaf (or other) cells and transportation to different plant organs. Thus, treatment of a plant which comprises a chimeric gene comprising a deacetylase coding sequence expressed in specific cells of the plant, will result in the conversion of N-acetyl PPT to PPT in those cells, causing these cells to die.

In a specific embodiment of the invention, the transgenic plant, comprising in its genome the DNA molecule of the invention encoding a protein having the biological activity of a deacetylase, additionally comprises, a chimeric gene conferring on said plants resistance a herbicide to PPT, such as, but not limited to the herbicide resistance genes, pat or bar described in EP-A-0 242 236, EP-A-0 242 246, EP-A-0 257 542. These genes encode enzymes which are capable of converting PPT into N-acetyl-PPT. This N-acetyl-PPT can be transported throughout the plant and act as the substrate for a deacetylase specifically expressed in certain plant cells or tissues. It is understood that according to this specific embodiment, induction of tissue-specific cytotoxicity will also occur upon treatment of the plant with PPT (EP-A-0 531 716, U.S. Pat. No. 5,668,297). Preferably, the deacetylase of the present invention and the enzyme conferring herbicide resistance gene will be expressed or transported to different cellular compartments (i.e. cytoplasm, chloroplast) to avoid re-conversion of the PPT to N-acetyl PPT, where this is not intended.

A chimeric marker gene as used herein is a gene comprising a marker DNA under the control of a plant-expressible promoter. The marker DNA encodes an RNA, protein or polypeptide, which, when expressed in a plant or plant cell, allows such plants or plant cells to be distinguished and separated from plants or plant cells not expressing the marker DNA. Examples of marker genes are genes that provide a specific color to the cells (such as the A1 gene, Meyer et al. 1987, Nature 330:667) or genes that encode a particular resistance to the plant cells or the plant such as antibiotic resistance (e.g. the aac(6′) gene, encoding resistance to gentamycin, WO94/01560).

Preferred marker genes are herbicide resistance genes. A herbicide resistance gene as used herein is a gene conferring to the plant or plant cell resistance to a herbicide. Examples of herbicide resistance genes are the genes encoding resistance to phenmedipham (such as the pmph gene, U.S. Pat. No. 5,347,047; U.S. Pat. No. 5,543,306), the genes encoding resistance to glyphosate (such as the EPSPS genes, U.S. Pat. No. 551047), genes encoding bromoxynyl resistance (such as described in U.S. Pat. No. 4,810,648) genes encoding resistance to sulfonylurea (such as described in EPA 0 360 750), genes encoding resistance to the herbicide dalapon (such as described in WO 99/27116), and genes encoding resistance to cyanamide (such as described in WO 98/48023 and WO 98/56238).

The cytotoxic effect of certain toxins may be enhanced by targeting to the specific parts of the cell, such as the chloroplast or mitochondria. Phosphinothricin is a glutamine synthase inhibitor. In plants, glutamine synthase exitst in multiple isozymic forms that can be localized within the cell in the cytosol and plastids. Thus, it can be contemplated that targeting of the deacetylase to the chloroplast, whereby N-acetyl-PPT is converted to PPT in the chloroplast, increases cytotoxic efficiency. Thereto, the foreign DNA comprising the deacetylase coding sequence can comprise an additional foreign DNA encoding a transit peptide. The additional DNA is preferably located between the first promoter and the deacetylase coding sequence. Alternatively, it can be desired that the marker protein or polypeptide is transported from the cytoplasm into chloroplasts or mitochondria of the transformed plant cells. Thereto, an additional DNA encoding a transit peptide is located between the marker DNA and its promoter. By transit peptide is meant a polypeptide fragment which is normally associated with a chloroplast or mitochondrial protein or subunit of the protein and is produced in a cell as a precursor protein encoded by the DNA of the cell. The transit peptide, is responsible for the translocation process of the nuclearencoded chloroplast or mitochondrial protein or subunit into the chloroplasts or mitochondria, and during such a process, the transit peptide is separated or proteolytically removed from the chloroplast or mitochondrial protein or subunit. One or more of such additional DNA's can be provided in the foreign DNAs of this invention as generally described and exemplified in European patent publication 0,189,707 and European patent EP 0 344 029.

Transformation of the plants of the invention can occur in a number of ways described in the art. Such methods include, but are not limited to Agrobacterium-mediated transformation (BP 0,116,718, DeBlaere: et al., 1987, Meth. Enzymol. 153:277-293), microinjection (Crossway et al., 1986, Mol. Gen. Genet. 102:179), direct gene transfer (Pazowski et al., 1984, EMBO J. 3:2717-2722), electrapomtion (Riggs et al., 1986, Proc. Natl. Acad. Sci. USA 83:5602-5606), or by particle gun bombardment (WO92/09696; Potrykus et al. 1991, Annu. Rev. Plant Physiol. Plant Mol. Biol. 42:205-225); see also Christou P. 1994, Agro-Food Industry Hi-Tech 2:17; Langridge et al. 1992, Plant J. 2:631; Vasil, 1994, Plant Mol. Biol. 25:925; Chen D., 1998, Methods Mol. Biol. 81:373. Alternatively, the foreign DNA comprising the chimeric genes of the invention can be inserted into the plastid genome of the cell or cells of a plant. Gene transfer can be carried out, for example, by particle gun bombardment (as described by Svab et al., 1990, Proc. Natl. Acad. Sci. USA 87:8526-8530) or by Agrobacterium-mediated transformation (De Block et al., 1985, EMBO J. 4:1367).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA sequences which is functionally or structurally defined, may comprise additional DNA sequences, etc.

Unless otherwise stated, all recombinant DNA techniques are carried out according to standard protocols as described in Sambrook et al. (1989) Molecular Cloning: A Laboratory Manual, Second Edition, Cold Spring Harbour Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R.D.D. Croy published by BIOS Scientific Publications Ltd (UK) and Blackwell Scientific Publications, UK.

In the description and examples, reference is made to the following sequences:

SEQ ID No. 1: Amino acid sequence of the ArgJ deacetylase from Comamonas acidovorans

SEQ ID No. 2: Nucleotide sequence encoding the Arg J deacetylase

### EXAMPLES

### Example 1

### Isolation of Bacterial Deacetylase Genes

The isolation of micro-organisms having N-acetyl-PPT deacetylase activity was performed as described in WO98/27201. Briefly, different soil samples were grown on Minimal Essential Medium containing only chitin as a carbon source. As chitin can only be broken down after deacetylation, microorganisms having deacetylase activity will preferentially grow on this medium. After subsequent replating and isolation of single colony bacteria, different colonies could be isolated that were capable of converting radioactive N-acetyl-PPT to PPT. One of the isolated bacteria was identified as Comamonas acidovorans and deposited at the Deutsche Sammlung fur Microorganismen und Zellkulturen under accession number DSM 11070.

In a next step the genes encoding for N-acetyl-PPT deacetylase activity were isolated. A specific selection method was developed to be able to isolate the deacetylase genes in a “Shot-gun” cloning method. The method is based on the principle that a gene product capable of deacetylating N-acetyl-PPT should also be able to process a related compound N-acetyl-Ornithine as a substrate. The deacetylation of N-acetyl-ornithine is a step in the arginine biosynthesis and mutants that are incapable of performing this step are auxotrophic for arginine. One such mutant, the E. coli nullmutant (XS1D2), which has a deletion in its ArgE gene, was described by Mountain et al. (1984, Mol. Gen. Genet. 197:82-89). Total DNA from Comamonas acidovorans was isolated, digested with EcoRI and ligated within the EcoRI site of the vector pACYC184 (Cang and Cohen, 1987, J Bacteriol. 134:1141-1156). The ligation mixture was then introduced by electroporation into the cells of the mutant E. coli XS1D2. Of 16.000 tested shot-gun clones, 14 were able to complement the arginine auxotrophy of the E. coli mutant. One of the complementing clones (E4.2) comprised a 4,2 kb EcoRI fragment. Hybridisation experiments confirmed that this fragment originated from the C. acidovorans genome and did not show any cross-hybridization with any of the other fragments in other clones.

Through subsequent subdloning, the minimal sequence for retaining complementation of the arginine auxotrophy was determined. This sequence was then sequenced as a double strand and the open reading frame was identified. Based on a homology with databank sequence, the sequence was termed as the ArgJ-gene.

### Example 2

### The ArgJ Coding Sequence of C. Acidovorans

Through subsequent subdloning, the minimal sequence for retaining complementation of the arginine auxotrophy was determined as a 2.2 kb HindIII/PstI fragment (SEQ ID No. 2). An open reading frame of 1230 bp was identified, encoding a protein of 410 amino acids (SEQ ID No. 1).

A potential ribosomal binding site is located at about 10 bp before the start-codon. Comparison of the amino acid sequence of the open reading frame with known sequences, strong homology was found with a number of N-acetyl-ornithin-transacetylases (Table 1). The strongest homology was found with N-acetyl-ornithin-transacetylase encoded by the ArgJ-gene from Neisseria gonorrhoeae. Therefore the complete open reading frame of the C. acidovorans fragment was named the ArgJ coding sequence. Upstream of the ArgJ coding sequence another, be it incomplete, open reading frame can be found. Comparison with databank sequences showed that this is part of the secA-gene of C. acidovorans. This sequence however, does not seem of importance for the complementation of the E. coli ArgE-mutant, since a PCR fragment comprising only the ArgJ-gene can restore arginin auxotrophy in the mutant.

TABLE 1
Protein sequences found to be homologous to the sequence
encoded by the C. acidovorans gene

| Homologous | | | |
|---|---|---|---|
| gene | Origin | Encoded protein | % Identical AA |
| ArgJ | Neisseria gonorrhea | N-acetyl-ornithin- | 54.5% |
| | | transferase | |
| ArgJ | Bacillus subtilis | N-acetyl-ornithin- | 37.4% |
| | | transferase | |
| ArgJ | Bacillus | N-acetyl-ornithin- | 37.5% |
| | stearothermophilus | transferase | |
| ArgJ | Saccharomyces | N-acetyl-ornithin- | 36.1% |
| | cervisiae | transferase | |

### Example 3

### Expression of the ArgJ Coding Sequence in E. coli

Through the selection procedure, it was established that the protein encoded by the ArgJ coding sequence from C. acidovorans was capable of complementing the E. coli ArgE mutant by its deacetylase activity. To establish whether this protein would also deacetylate N-acetyl-PPT, both coding sequences were over-expressed in E. coli, and the supernatant of the culture was analyzed for its ability to deacetylate N-acetyl-PPT.

The coding sequence was cloned into a vector with high copy number under control of the lacZ promoter. The incorporation of a stop codon made overexpression of the deacetylase sequence possible without interference of open reading frames of the lacZ gene. Compared to the isolated fragments, these construct showed significantly stronger complementation of the E. coli ArgE mutant.

### Example 4

### Expression of the ArgJ Coding Region from C. acidovorans in Tobacco

The ArgJ coding sequence was recloned into the binary vector pROK1 under control of the Cauliflower Mosaic Virus 35S promoter sequence. The resulting plasmid p35SARG comprising the 35S promoter, ArgJ coding sequence and 35S terminator was transformed in Agrobacterium tumefaciens (strain ATHV) using standard methods. Tobacco (Nicotiana tabacum) leaf disks were transformed with the recombinant Agrobacteria according to the method of Horsch et al. (1985, Science 227:1229-1231) and selected on kanamycin medium. Seven independent transformants were regenerated. In a drop experiment, the leaves of the transgenic plants (and a number of control plants) were treated with 5 μl each of the following concentrations of N-acetyl- D, L-PPT: 4 mg/ml (=15 mM), 1 mg/ml (=3.75 mM), 0.4 mg/ml (=1.5 mM), 0.1 mg/ml (=0.38 mM). The treatment sites were examined for lightening or necrosis formation. Four plants showed clearly visible leaf damage, apparent 4 to 5 days after treatment with a dose of 4 mg/ml or 1 mg/ml.

### Example 5

### Tapetum-Specific Expression of the ArgJ Gene in Tobacco

The ArgJ coding sequence was recloned into the binary vector pROK1 under control of the TA29 promoter of tobacco. The resulting plasmid pTA29ARG comprising the TA29 promoter, ArgJ coding sequence and 35S terminator was transformed in Agrobacterium tumefaciens (strain ATHV) using standard methods. Tobacco (Nicotiana tabacurn) leaf disks were transformed with the recombinant Agrobacteria according to the method of Horsch et al. (1985, above) and selected on kanamycin medium.

To investigate tapetum-specific expression of the ArgJ deacetylase, flower buds of the transgenic lines were treated with N-acetyl-PPT to induce male-sterile flowers.

In each case only one side of the inflorescence (the total area of the developing flower bud) was treated with N-acetyl PPT, in order to eliminate possible contamination with natural male-sterility phenomena. Treatment was carried out on flower buds of 2 to 4 mm large. Transgenic and control plants were treated with 10 μl of 5 mg/ml N-acetyl PPT solution (0.1% Genapol as wetting agent). After the blooming of the buds (about 9-11 days after conclusion of the treatment) the plants were investigated for the occurrence of male-sterile flowers.

As expected, no reaction was seen in either the control or the wild-type tobacco flowers. In three out of seven transgenic plants, male-sterile flowers developed from the drop-treated flowerbuds. The anthers of these flowers did not contain any pollen and their filaments were markedly shorter than those of treated wild-type flowers. Anther dehiscence occurs in these flowers, but is delayed compared to controls. If cross-pollination is avoided, the male-sterile flowers develop an empty seedpod, as pollen for self-pollination is lacking. Otherwise these flowers can not be distinguished from untreated flowers.

1. A DNA molecule encoding a protein or polypepetide having deacetylase activity, wherein said protein has at least 70% to 75% sequence indentity with the sequence of SEQ ID No. 1.

2. The DNA molecule of claim 1, wherein said protein or polypeptide comprises the sequence of SEQ ID No. 1.

3. The DNA molecule of claim 1, comprising a nucleotide sequence which has at least 70% to 75% sequence identity to the sequence of SEQ ID No. 2.

4. The DNA molecule of claim 3, comprising the nucleotide sequence of SEQ ID No. 2.

5. The DNA molecule of claim 1, which can be isolated from Comamonas acidovorans, deposited as DSM 11070.

6. A chimeric gene comprising:
a) the DNA molecule of any one of claims 1 to 5,
b) a plant-expressible promoter; said DNA molecule being in the same transcriptional unit and under the control of said plant-expressible promoter.

7. A plant having plant parts which can be destroyed upon induction, said plant comprising, stably integrated in its genome, a chimeric gene, comprising
a) The DNA molecule of any one of claims 1 to 5,
b) a plant-expressible promoter; said DNA molecule being in the same transcriptional unit and under the control of said tissue-specific, plant-expressible promoter;
wherein said plant is characterized by the fact that, upon treatment with N-acetyl-PPT, plant parts are destroyed.

8. The plant of claim 7, wherein said plant-expressible promoter is a tissue-specific promoter.

9. The plant of claim 8, wherein said tissue-specific promoter is a stamen selective promoter;

10. A plant which is inducibly male-sterile, said plant comprising, stably integrated into its genome, a foreign DNA comprising a chimeric gene comprising:
a) the DNA molecule of any one of claims 1 to 5; and
b) a promoter directing stamen-selective expression; said DNA being in the same transcriptional unit and under the control of said stamen-selective promoter.

11. The plant of claim 10, wherein said stamen-selective promoter is selected from the group of: the TA29 promoter, the T72 promoter, the El promoter, or the CA55 promoter.

12. The plant of claim 10, wherein said foreign DNA further comprises:
a herbicide resistance gene comprising a DNA encoding an RNA, protein or polypeptide that confers herbicide resistance on said plant.

13. A process for producing a plant with selectively destroyable plant tissues or parts, said process comprising:
i) introducing into a plant cell or tissue a foreign DNA comprising a chimeric gene comprising:
a) The DNA molecule of any one of claims 1 to 5,
b) plant-expressible promoter; said DNA molecule being in the same transcriptional unit and under the control of said plant-expressible promoter.
ii) regenerating said plant from said plant cell.

14. The process of claim 13, wherein said plant-expressible promoter is a tissue-specific promoter.

15. The process of claim 14, wherein said tissue-specific promoter is a male or female organ selective promoter and said plants are inducibly male or female sterile.
